(19) United States
(12) Patent Application Publication
(10) Pub. No.: US 2024/0408271 A1
(43) Pub. Date: Dec. 12, 2024

(54) Functionalized Linkers in Responsive Biomaterials

(71) Applicant: University of Cincinnati, Cincinnati, OH (US)

(72) Inventors: John Robert Martin, Cincinnati, OH (US); Karina Ann Bruce, Findlay, OH (US); Dylan Widder Marques, Cincinnati, OH (US); Alan Joseph Fullenkamp, Beavercreek, OH (US)

(73) Assignee: University of Cincinnati, Cincinnati, OH (US)

(21) Appl. No.: 18/699,494

(22) PCT Filed: Oct. 11, 2022
(86) PCT No.: PCT/US2022/046285

§ 371 Date: Apr. 8, 2024

Related U.S. Application Data
(60) Provisional application No. 63/254,041, filed on Oct. 8, 2021.
(60) Provisional application No. 63/254,044, filed on Oct. 8, 2021.
(60) Provisional application No. 63/254,045, filed on Oct. 8, 2021.

(51) Int. Cl. A61L 27/18; A61L 27/52

(52) U.S. Cl. CPC A61L 27/18; A61L 27/52

(57) ABSTRACT

A method of incorporating one or more linkers into a biomaterial with one or more ketone units is disclosed. The method involves reacting a linker-containing material selected from the group consisting of thiol-containing compositions, thioketal-containing compositions, selenol-terminated compositions and combinations thereof with the biomaterial in the presence of an acid catalyst.

### CROSS-REFERENCE TO RELATED APPLICATIONS

This application is a continuation of PCT Application No. PCT/US22/46285 filed Oct. 11, 2022, which claims the benefit of the filing date of U.S. Provisional Application No. 63/254,041 filed Oct. 8, 2021, U.S. Provisional Application No. 63/254,044 filed Oct. 8, 2021, and U.S. Provisional Application No. 63/254,045 filed Oct. 8, 2021, the disclosures of which are incorporated by reference herein in their entireties.

### TECHNICAL FIELD

The present invention relates to methods for incorporating linkers into biomaterial.

### BACKGROUND OF THE INVENTION

Environmentally responsive polymers that can tie programmed material functionality to local biological stimuli have garnered increasing interest in regenerative medicine applications. These activatable systems are specifically triggered by biological signals such as cell-generated reactive oxygen species (ROS). Among many potential medicinal uses, responsive biomaterial implants can release therapeutics to select tissue areas to limit off-target effects or selectively degrade when in contact with new cellular growth. Polymer systems featuring thiol-containing and thioketal (TK) linkers have been particularly popular as ROS-responsive materials due to their case of synthesis, insensitivity to hydrolysis even at extremely acidic or basic pH levels, and selective degradation when exposed to ROS. However, conventional TK units are relatively hydrophobic and struggle to rapid responsivity to physiologic ROS doses when employed in vivo.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Embodiments of the disclosed invention are directed to a method of incorporating one or more linkers into a biomaterial with one or more ketone units. The method involves reacting a linker-containing material selected from the group consisting of thiol-containing compositions, thioketal-containing compositions, selenol-terminated compositions and combinations thereof with the biomaterial in the presence of an acid catalyst. In one embodiment, the material is a thiol-containing composition. In another embodiment, the thiol-containing composition is cysteamine. In one embodiment, the material is a selenol-terminated composition. In another embodiment, the selenol-terminated composition is 2-amino ethaneselenol.

In one embodiment of the present invention, the material is a thioketal-containing composition. In another embodiment, the thioketal-containing composition is cysteamine. In one embodiment, the thioketal-containing composition is ethyl pyruvate. In another embodiment, the linker in the linker-containing material is aromatic. In one embodiment, the linker in the linker-containing material is cationic. In another embodiment, the linker in the linker-containing material is anionic. In one embodiment, the linker in the linker-containing material is a sulfonyl linker.

In another embodiment of the present invention, the biomaterial is selected from the group consisting of polymeric coatings, tissue engineering scaffolds, hydrogels, and nanoparticles. In one embodiment, the acid catalyst is selected from the group consisting of p-toluene sulfonic acid, tifluoroacetic acid, bismuth chloride, hydrochloric acid, and sulfuric acid. In another embodiment, the reaction is run under a nitrogen atmosphere. In one embodiment, the reaction is run with heat.

### BRIEF DESCRIPTION OF THE DRAWINGS

The objects and advantages of the disclosed invention will be further appreciated in light of the following detailed descriptions and drawings in which:

FIG. 1 is a synthesis scheme for thioketal linkers with conjugated small molecule drug compounds.

FIG. 2 is a synthesis scheme for a library of selenoketal linkers

FIG. 3A is a synthesis scheme for a library of functionalized thioketal linkers

FIG. 3B is a list of linkers useful in the scheme shown in FIG. 3A.

FIG. 4A is an image of the chemical structure for the antibiotic ciprofloxacin. It has a Log P of 1.32.

FIG. 4B is an image of the chemical structure for the NSAID tolmetin. It has a Log P of 2.07.

FIG. 4C is an image of the chemical structure for the hormone progesterone. It has a Log P of 3.78.

FIG. 5 an image of three graphs showing the ROS-mediated liberation of the small molecule drug tolmetin from a TK drug conjugate as assessed by HPLC.

FIG. 6A is a schematic overview outlining conventional thioketal bond chemistry.

FIG. 6B is a schematic overview outlining strategies of the present invention for re-engineering these linkers to enhance oxidative sensitivity and functionality.

FIG. 7A is a graph showing ROS dose-responsiveness of hydrophobic PTK-urethane scaffolds.

FIG. 7B is a graph showing ROS dose-responsiveness of PTK hydrogels.

FIG. 7C is a graph showing that conformal PTK polymer coatings all display limited reactivity in purely aqueous environments but significant reactivity to increasing doses of ROS (*p<0.05). However, ROS responsiveness correlates with material hydrophilicity (scaffold<hydrogel<polymer coating) and only the nanoscale PTK coating achieves significant (though minimal) triggering at physiologic 0.1 mM H₂O₂.

FIG. 8 is an NMR of a model selenoketal linker.

FIG. 9 is a graph showing SK and TK linker degradation over time when incubated in 10 mM H₂O₂.

FIG. 10A is an NMR spectrum of a base TK linker.

FIG. 10B is an NMR spectrum of a Pyr-TK, linker.

FIG. 10C is an NMR spectrum of a Lev-TK linker.

FIG. 11A is a graph showing TK linker reduction in bond persistence over time when incubated in 1M H₂O₂.

FIG. 11B is a graph showing TK linker degradation over time when incubated in 1M H₂O₂.

FIG. 12 is a synthesis scheme for the thioketal linker synthesized from a protected cysteamine monomer and the antioxidant drug ethyl pyruvate.

FIG. 13A is an NMR spectrum of EPTK after synthesis

FIG. 13B is an NMR spectrum of the thioketal peak after three days of degradation in varying ROS concentrations

FIG. 13C is a graph showing the HPLC of ethyl pyruvate.

FIG. 13D is a graph showing the HPLC of degraded EPTK.

FIG. 13E is a graph of a degradation study that shows release of ethyl pyruvate from EPTK over time at varying ROS concentrations.

FIG. 14A is a graph showing in vitro testing with MC3T3-E1 pre-osteoblast cells to establish cytotoxicity of hydrogen peroxide.

FIG. 14B is a graph showing in vitro testing with MC3T3-E1 pre-osteoblast cells to establish cytotoxicity of ethyl pyruvate.

FIG. 14C is a graph showing a cell saving study capability test of released ethyl pyruvate and degraded ethyl pyruvate.

### DEFINITIONS

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “linker” means a crosslinker or cross-linking agent containing at least two functional groups.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Though small molecule compounds are the most common class of medicinal therapeutics, their translation into localized drug delivery applications can be surprisingly challenging. Since these low molecular weight compounds are almost all designed with high aqueous solubility for systemic administration, it is difficult to retain these highly diffusible drugs in an implanted biomaterial matrix for controlled therapeutic delivery. Considering these obstacles, covalent conjugation of small molecule drugs to polymeric implants has emerged as an attractive strategy for achieving localized therapeutics release. However, current drug/polymer conjugates suffer from a number of significant drawbacks: conventional molecular conjugation handles (alcohols, amines, thiols, carboxylic acids) are not present in all drug compounds, linker degradation often leaves inhibitory residual byproducts on discharged drug molecules, and hydrolytic cleavage of most conventional drug linkers is minimally-responsive to local biological factors. In one embodiment, the present invention involves a simple drug/polymer conjugation that can selectively release intact drug molecules upon specific triggering by local tissue.

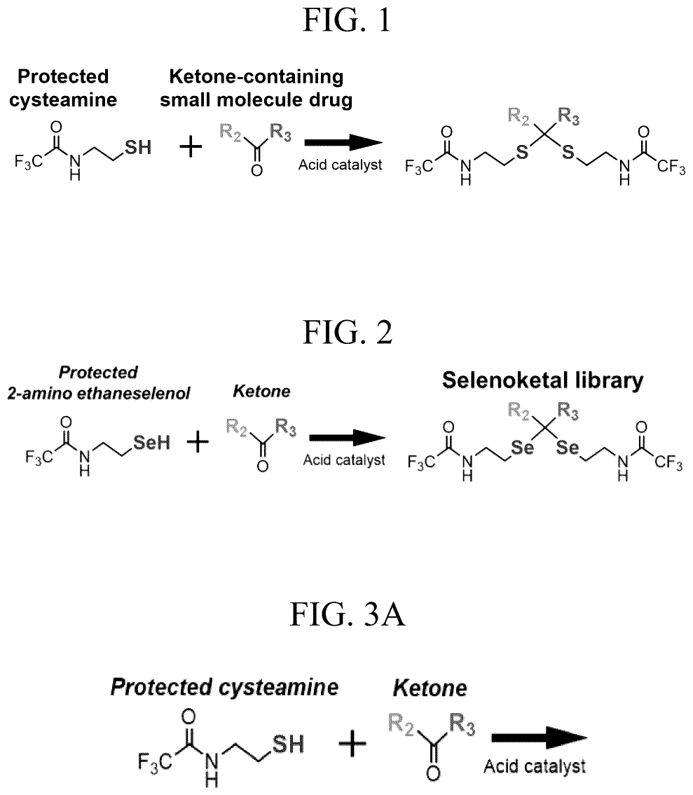

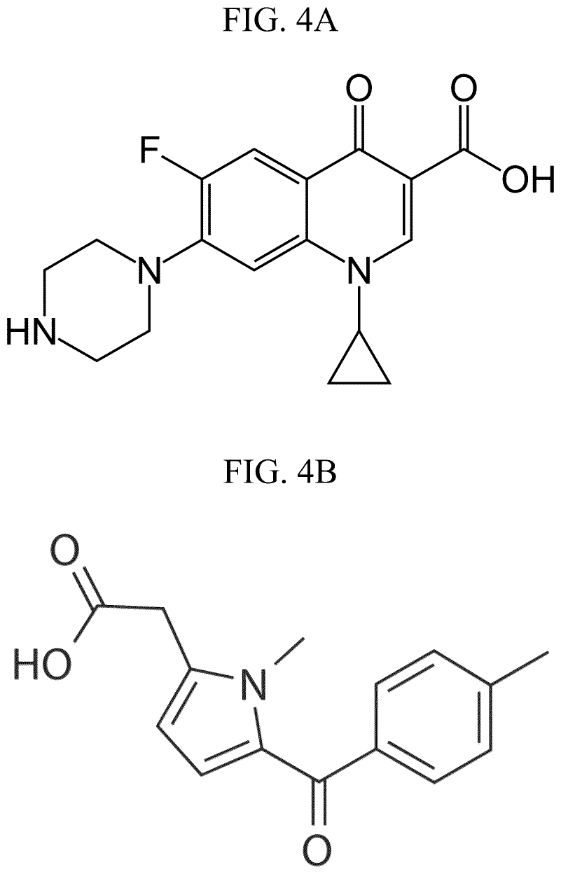

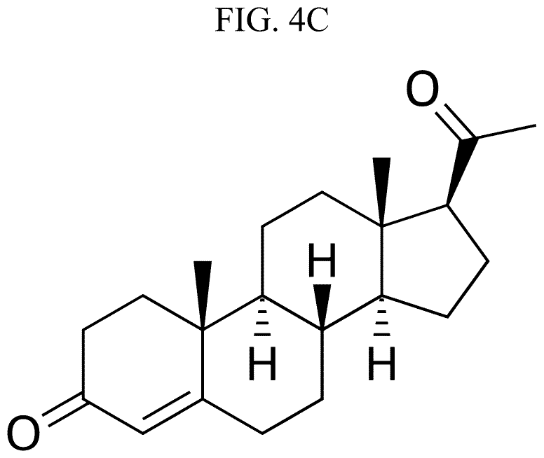

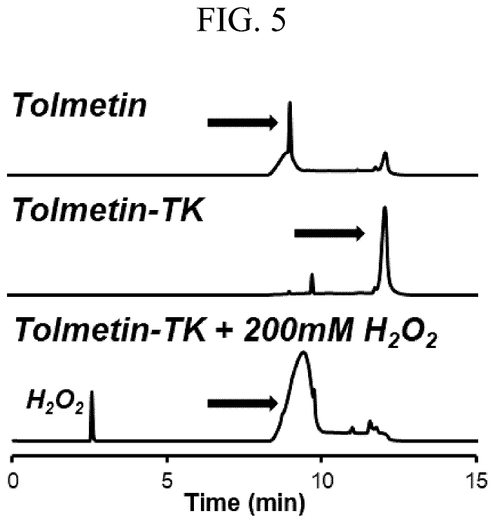

Before their employment in various biomaterials, thioketal units were originally developed as simple protecting groups for ketone groups in organic synthesis methods. TK bonds regenerate their original ketone structure upon oxidation, and this unique chemical behavior motivates a new strategy for small molecule drug conjugation and triggerable release using thioketal-linked materials. In one embodiment, the present invention condenses thiol-containing precursors around a drug molecule's ketone unit to form an ROS-cleavable TK bond within a polymerizable drug conjugate monomer as demonstrated in FIG. 1. This approach sequesters small molecule drugs within a larger polymer structure, provides the biomaterial with oxidation-responsive drug release capacity, and regenerates the original drug molecule upon triggered release. Candidate drug molecules featuring ketone moieties are listed in FIGS. 4A-4C, and ROS-triggered release of the small molecule drug tolmetin from a TK conjugate was confirmed using high performance liquid chromatography (HPLC) as shown in FIG. 5. In short, this technology presents a new technique for localized and responsive drug delivery for implantable biomaterial.

This embodiment of the present invention provides numerous benefits. The benefits include easy and inexpensive synthesis of new drug-conjugated TK linkers using simple condensation reactions between thiolated precursors and ketone-containing molecules. In addition, polymer-drug conjugates can be generated that release original drug compounds upon treatment with ROS. Among other uses, these materials can be used to make injectable tissue engineering scaffolds or conformal drug coatings.

In another embodiment, the present invention involves the incorporation of selenoketal (SK) bonds into biomaterial systems to serve as more responsive analogues to conventional thioketals. The synthetic scheme for these novel SK linkers is outlined in FIG. 2. The figure describes SK bond formation through the condensation of selenol-terminated precursors with the ketone units on respective linking molecules.

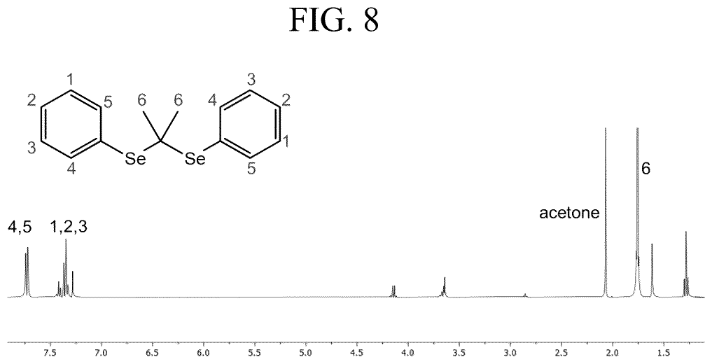

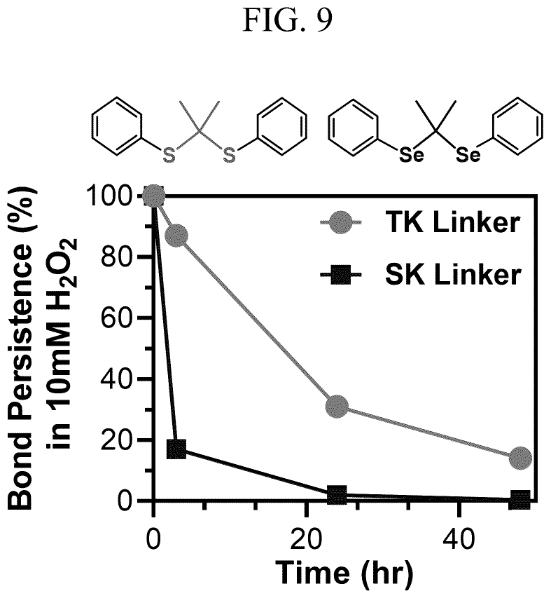

The nuclear magnetic resonance (NMR) spectra of a model SK compound is shown in FIG. 8 and confirms the successful synthesis of this material. When incubated in a model oxidative environment of 10 mM hydrogen peroxide (H₂O₂) and evaluated by NMR over time, the SK compound also demonstrated significantly enhanced degradation compared to a TK analogue as shown in FIG. 9. These NMR experiments also showed that the novel SK linker is unchanged when incubated in non-oxidative aqueous media, demonstrating the selectivity of its responsiveness like previously demonstrated TK materials. These SK-linked materials are expected to be used in the fabrication of systems, including tissue engineering scaffolds and polymeric drug coatings.

This embodiment of the present invention provides numerous benefits. The benefits include easy and inexpensive synthesis of new SK linkers using simple condensation reactions between selenol precursors and ketone-containing molecules. In addition, SK sensitivity to oxidative degradation is increased compared to benchmark TK linkers. Among other uses, these materials can be used to make injectable tissue engineering scaffolds or conformal drug coatings.

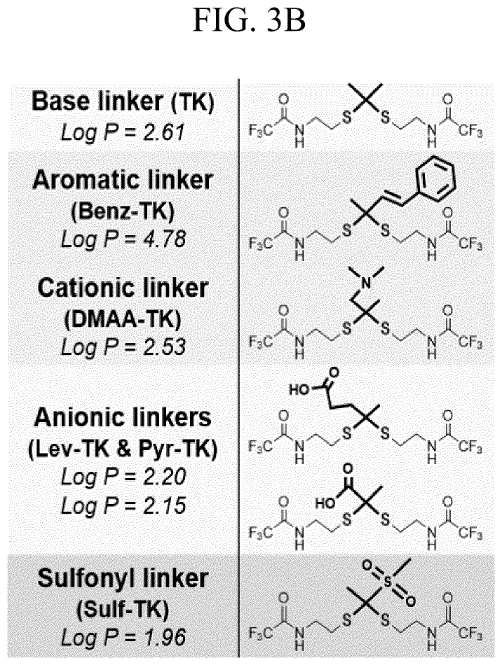

In another embodiment of the present invention, novel TK linkers are disclosed that move beyond the standard TK chemistry and feature more complex molecules in the linker structure. These include ionizable carboxylic acids or tertiary amines, aromatics, and sulfonyl groups. The synthetic scheme for these novel linkers is outlined in FIG. 3A, and a library of linkers are shown in FIG. 3B. The figures describe TK bond formation through the condensation of thiolated precursors with the ketone units on respective linking molecules.

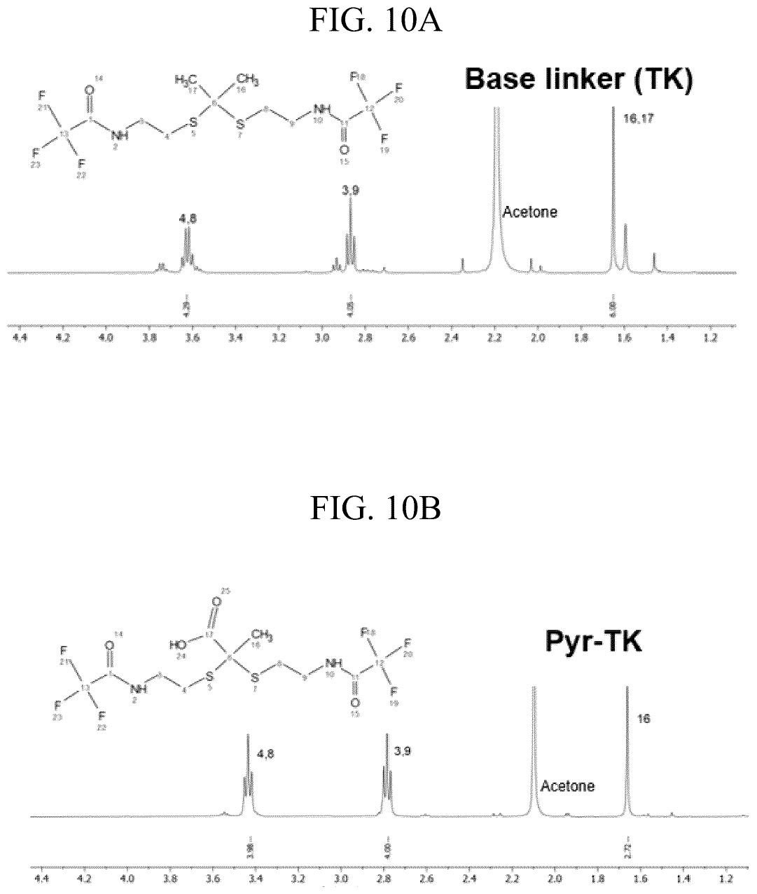

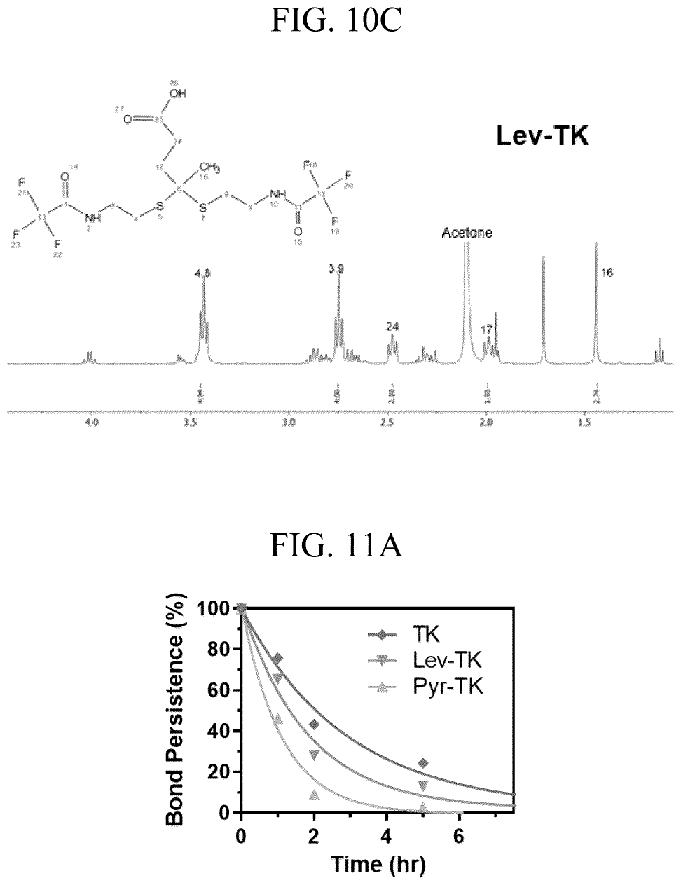

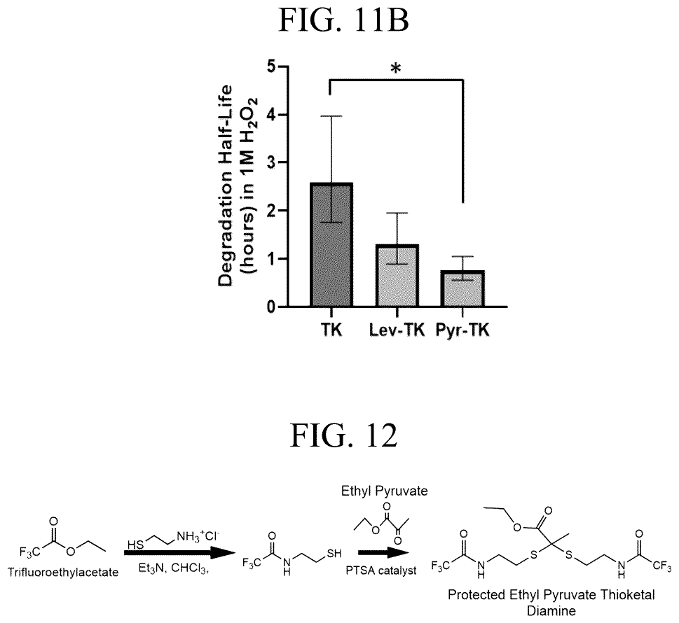

The nuclear magnetic resonance (NMR) spectra of the base TK, Lev-TK, and Pyr-TK materials are given in FIGS. 10A-10C and confirm the successful synthesis of these molecules. When incubated in a model oxidative environment of 1M hydrogen peroxide (H₂O₂) and evaluated by NMR over time, these compounds also demonstrated differential cleavage kinetics of their thioketal bonds as shown in FIGS. 11A and 11B. The most hydrophilic Pyr-TK linker (predicted Log P value of 2.15) demonstrated a significant decrease in degradation half-life compared against the more hydrophilic base TK formulation (Log P of 2.61). These data indicate that by increasing TK bond hydrophilicity, TK susceptibility to oxidative cleavage is increased. These NMR experiments also showed that all three TK linkers are unchanged when incubated in non-oxidative aqueous media, demonstrating the selectivity of their responsiveness. Moreover, the incorporation of pyruvic or levulinic acid into the TK bond structure creates linkers with free carboxylic acid groups and an anionic character. This innovation allows for the creation of ionizable polymer structures for a host of applications in biomaterial-based technologies

This embodiment of the present invention provides numerous benefits. The benefits include easy and inexpensive synthesis of new TK linkers using simple condensation reactions between thiolated precursors and ketone-containing molecules. In addition, new TK linkers can be generated with varied hydrophilicity, incorporation of ionizable units, and increased sensitivity to oxidation. Among other uses, these materials can be used to make injectable tissue engineering scaffolds or conformal drug coatings.

### Stimuli-Responsive Biomaterials in Regenerative Medicine

Biomaterial implants fabricated from synthetic polymers have been extensively used in regenerative medicine applications and are regularly formulated into erodible drug delivery systems or degradable scaffolds. In vivo degradation of these synthetic implants is most commonly facilitated by hydrolysis of ester bonds in the polymer structure and can also be simply modulated by tuning polymer crystallinity or hydrophilicity. Though implant hydrolysis mediated by the body's aqueous environment is effective in many applications, this material biodegradation strategy is minimally-responsive to changes in local tissues. Subsequently, these materials rely on pre-determined degradation rates encoded in the original formulation that may imperfectly translate into an innately heterogenous patient population. The resulting mismatch between rates of implant resorption and tissue regeneration can compromise overall healing, whether from prematurely degraded tissue engineering scaffolds or drug-loaded implants that prematurely released their therapeutic payloads. To this end, environmentally-responsive polymers have been extensively investigated as “smart” materials that react to specific biological stimuli, including enzymes, pH, or reactive oxygen species. Prominent examples of stimuli-responsive biomaterials include hydrogels selectively degraded by specific cell-produced proteinases, gels with pH-responsive drug release to target inflamed tissues, and hydrogels with oxidation-triggered release of small molecule compounds.

Due to their selective production by cells and their elevated presence in healing tissues, ROS such as hydrogen peroxide (H₂O₂), superoxide, hydroxyl radical, or hypochlorite are particularly attractive signals for facilitating specific biomaterial responsiveness. These highly reactive and short-lived molecules are important mediators in various biological processes and the immune response, and elevated ROS, or “oxidative stress”, is a hallmark characteristic of inflammation and pathogenesis in many diseases. Many synthetic polymers with oxidation responsiveness have been developed over the past two decades, including polysulfides, selenium-linked polymers, poly(oxalates), phenylboronic esters, oligoprolines, and thioketals. These materials have been successfully employed both as triggerable nano-scale drug delivery vehicles and bulk-scale biodegradable implants. Despite the exciting promise of responsive polymer systems for a number of biomedical applications, limitations in material sensitivity, specificity, and speed of responsiveness to relatively scarce biological signals remain the central bottleneck in their continued development. Developing reactive polymer systems that can quickly respond to biologically-relevant concentrations of triggering stimuli remains a critical goal in both fundamental and translational biomaterials research.

### Thioketal Linkers in Oxidation-Sensitive Polymer Systems

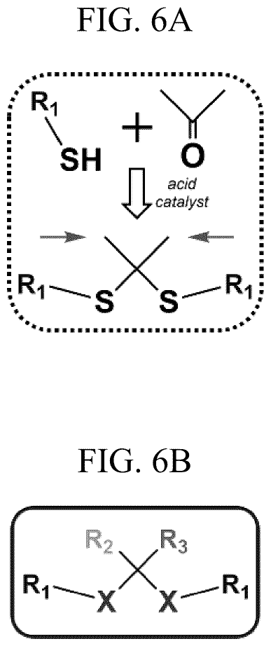

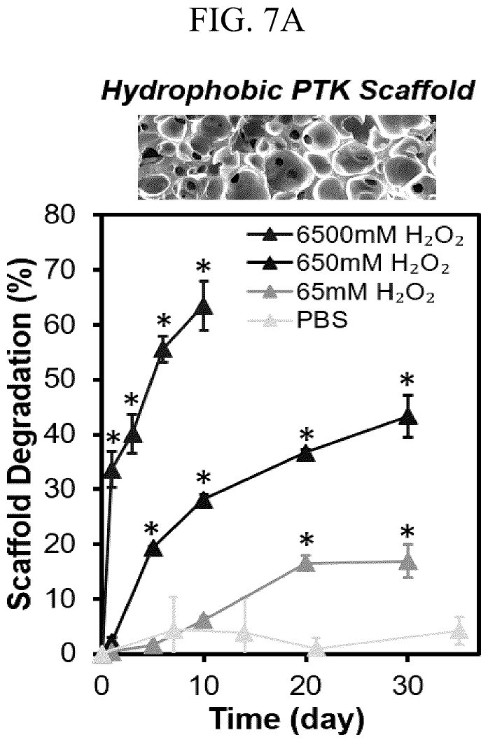

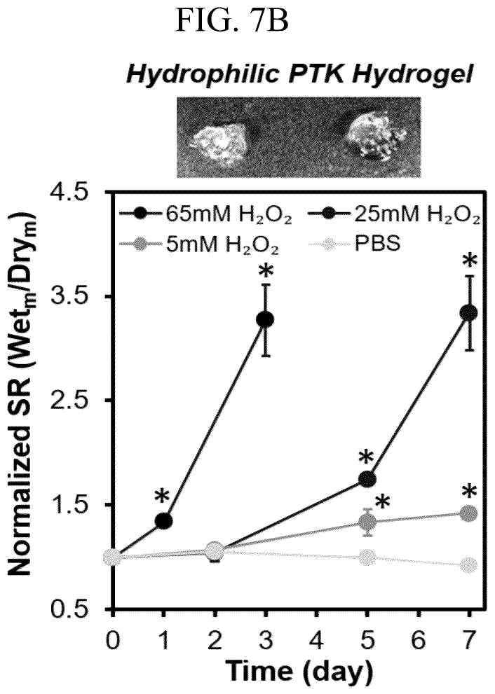

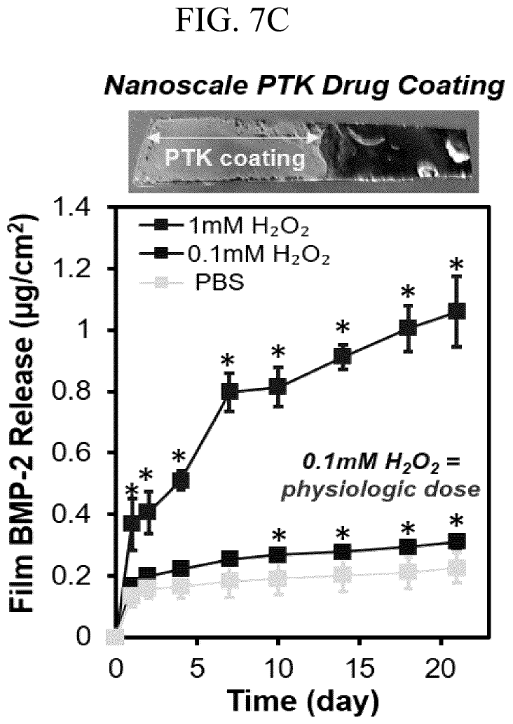

Since their introduction by the Murthy group in 2010, thioketal linkers have been incorporated into a number of polymeric biomaterial systems to confer ROS responsiveness. Conventional TK bonds (general structure given in FIG. 6A) are nearly completely insensitive to hydrolysis even at extremely acidic or basic pH levels, but are selectively cleaved when exposed to ROS. This selective polymer degradation mechanism has typically been employed to target tissues producing high levels of ROS such as intestinal lesions, inflamed muscles, or cancer cells with triggerable nano-therapies. Efforts pioneered by the PI have also translated new poly(thioketal) (PTK) polymer formulations into bulk-scale biomaterials for a host of tissue engineering applications. These previously described systems have featured degradable TK linkers in the respective polymeric structures to mediate selective responsiveness. They are all nearly completely inert in purely aqueous conditions but display dose-responsive behavior when incubated with ROS. These include PTK-urethane scaffolds (FIG. 7A) that facilitated cell-mediated material resorption and tissue regeneration in skin wounds4, PTK-crosslinked poly(ethylene glycol) (PEG) hydrogels (FIG. 7B) for injectable stem cell delivery from a cell-degradable synthetic matrix, and conformal PTK coatings (FIG. 7C) for on-demand delivery of the film-encapsulated protein drug bone morphogenetic protein-2 (BMP-2) to bone defects. In short, employing the simple yet versatile TK chemistry is a highly promising strategy for creating stimuli-responsive biomaterial systems.

However, polymers with TK linkers do feature significant hurdles that must be overcome to facilitate their translation into clinically-viable technologies. The most significant challenge remains the relatively limited responsiveness of TK-based materials to physiologically-relevant doses of ROS, which are quantified at less than 0.1 mM in most tissues and feature in vivo half-life values ranging from micro- to nano-seconds. Though all the PTK systems highlighted in FIGS. 7A-7C indicate robust ROS dose responsiveness in benchtop testing, it must be noted that only the nanoscale drug coatings in FIG. 7C achieved significant (though moderate) in vitro responsiveness in 0.1 mM H₂O₂. Consequently, though all three of these formulations displayed some level of in vivo responsiveness, their activity only significantly manifested after weeks or months of material implantation. While prolonged material retention can be beneficial for scaffolds that promote soft tissue infiltration or hydrogels that protect encapsulated cells from immunotoxicity, many other biomaterial applications require significantly increased reactivity. Recent reports further highlight this need. The ROS-degradable drug coatings developed by the inventors facilitated less bone growth than expected due to too-slow BMP-2 release kinetics in vivo, while PTK-based bone cements produced by Guelcher et al. featured negligible in vivo degradation and remodeling in slow-healing rat and rabbit bone defect models. In short, increasing the oxidative sensitivity of TK-based materials would represent a significant breakthrough for this technology.

Improving the responsiveness of ROS-responsive biomaterials through polymer engineering strategies has been explored in other systems, but essentially all TK-based technologies to date feature the same conventional linker structure described in FIG. 6A. Crucial work from the Thayumanavan group recently elucidated the exact mechanism by which oxidative species facilitate TK bond cleavage, and also attempted to describe the relationship between bond structure and oxidative sensitivity. While this report provided numerous valuable findings, the lead-candidate TK structure from this work still maintained the same basic configuration and ROS responsiveness as the many previously-described TK-linked biomaterials. However, the present inventor's depth of experience with these systems has generated additional insight into their behavior; as demonstrated in FIGS. 7A-7C, increasing material hydrophilicity (nanoscale coating>hydrogel>scaffold) also increases material oxidative responsiveness. It is hypothesized that the inherently hydrophobic TK bond structure (featuring two non-polar pendant methyl groups) limits contact with ROS in aqueous environments both in vitro and in vivo. In short, increasing material hydrophilicity boosts ROS interactions with the degradable linker to improve stimuli responsiveness. This also extends to nano-scale PTK polymer systems, which possess extremely high surface area to volume ratios. This likely increases material interactions with water-borne ROS to consequently increase sensitivity to oxidation. While creating more hydrophilic polymer networks surrounding TK linkers remains a viable approach to increase ROS responsiveness, a more widely-translatable and effective strategy would be to re-engineer the degradable linker itself (FIG. 6B). The present invention uses new configurations of oxidation-sensitive bonds to create materials that are highly responsive to biologically-relevant levels of ROS.

### Selenium-Based Biomaterials

Residing below sulfur in group 16 on the periodic table, selenium shares many common attributes with this elemental building block found in TK units. These parallels include similar methodologies for incorporating these elements into organic molecules, and responsiveness to oxidation as reviewed in previous reports. However, since the lower electronegativity of selenium compared to sulfur creates weaker bond energies with carbon (C—S 272 KJ/mol; C—Se 244 KJ/mol), selenium-based biomaterials also feature enhanced sensitivity to oxidation. Much like conventional polysulfide biomaterials, selenic polymers have primarily been employed in nanoparticles for targeted drug delivery applications. These systems typically rely on a hydrophobic-to-hydrophilic phase change (i.e. minimal covalent bond scission) upon selenium oxidation to facilitate material responsiveness. Though phase inverting polymers can be highly useful in nano-scale colloids, their lack of covalent degradation does limit their applicability. High molecular weight polymers can have difficulty effectively clearing from the body through renal filtration, and phase inversion methods of material biodegradation are broadly incompatible with resorbable polymer systems featuring covalent crosslinks. To this end, bulk-scale polymers used in tissue engineering applications typically feature fully cleavable linkers to generate low molecular weight species upon in vivo implant degradation.

Polymer systems with sulfur-based thioketal linkers undergo ROS-mediated chain cleavage to facilitate in vivo material resorption. Selenoketal (SK) bonds, which have been previously described but never employed in biomedical applications, are selenium-based analogues to TK linkers (FIG. 6B). Consequently, they should achieve full covalent cleavage upon oxidation while maintaining stability in non-oxidative conditions. However, like previously described selenium-containing biomaterials, SK-based materials are expected to feature significantly increased ROS sensitivity compared to conventional TK polymers. Using SK linkers in responsive polymer systems leverages the benefits of highly-reactive selenic polymers with the added advantage of functioning in a fully degradable configuration. This embodiment of the present invention has the potential to greatly enhance the efficacy of responsive polymer systems by creating materials with acute sensitivity to physiologic levels of ROS.

### Polymer/Drug Conjugates as Therapeutic Delivery Vehicles

Though small molecule compounds are the most common class of medicinal therapeutics, their translation into localized drug delivery applications can be surprisingly challenging. Since these low molecular weight compounds are almost all designed with high aqueous solubility for systemic administration, it is difficult to retain these highly diffusible drugs in an implanted biomaterial matrix for controlled therapeutic delivery. Considering these obstacles, covalent conjugation of small molecule drugs to polymeric implants has emerged as an attractive strategy for achieving localized therapeutics release. Some notable examples include tunable dexamethasone release from polymer conjugates for the treatment of arthritic rat joints, and sustained delivery of the anti-inflammatory drug diclofenac from polymer conjugates encapsulated within an electrostatic implant coating. However, current drug/polymer conjugates do suffer from a number of significant drawbacks: conventional molecular conjugation handles (alcohols, amines, thiols, carboxylic acids) are not present in all drug compounds, linker degradation often leaves inhibitory residual byproducts on discharged drug molecules, and hydrolytic cleavage of most conventional drug linkers is minimally-responsive to local biological factors. Consequently, simple drug/polymer conjugation that can selectively release intact drug molecules upon specific triggering by local tissue is still needed.

Before their employment in various biomaterials, thioketal units were originally developed as simple protecting groups for ketones in organic synthesis methods. TK bonds can be selectively cleaved by oxidation to regenerate the original ketone structure, with this phenomenon also extending to TK linkers in biomaterial systems which generate their acetone precursor upon oxidation. This unique chemical behavior motivates our exploration of a new strategy for small molecule drug conjugation and triggerable release using these materials. Herein, we propose to condense thiol (or selenol)-containing precursors around a drug molecule's ketone unit to form an ROS-cleavable TK/SK bond within a polymerizable drug conjugate monomer. This approach fulfills numerous design criteria: sequestering small molecule drugs within a larger polymer structure, providing the biomaterial with oxidation-responsive drug release capacity, and regenerating the original drug molecule upon triggered release. To date, related strategies using comparable chemistries have been explored in a few systems to deliver the small molecule cinnamaldehyde or antimicrobial compound p-anisaldehyde, indicating the general feasibility of the proposed approach. However, the development of polymer systems conjugated to ketone-containing small molecule drugs through oxidation-sensitive linkers has not been demonstrated. The present invention both expands the functionality of ROS-degradable biomaterials and presents a new technique for localized drug delivery for regenerative applications.

### EXAMPLES

### Example 1

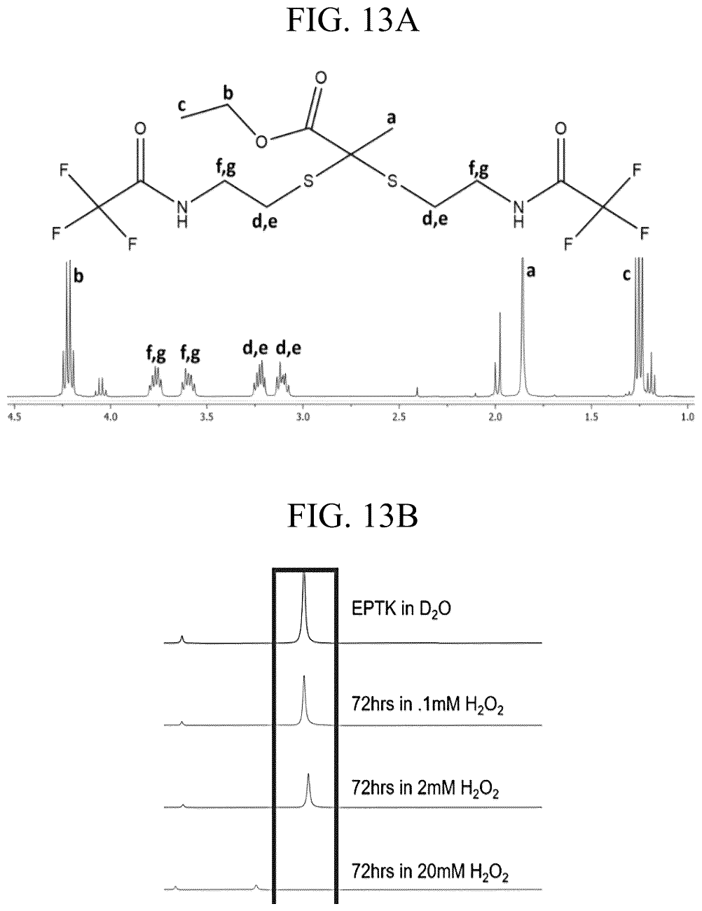

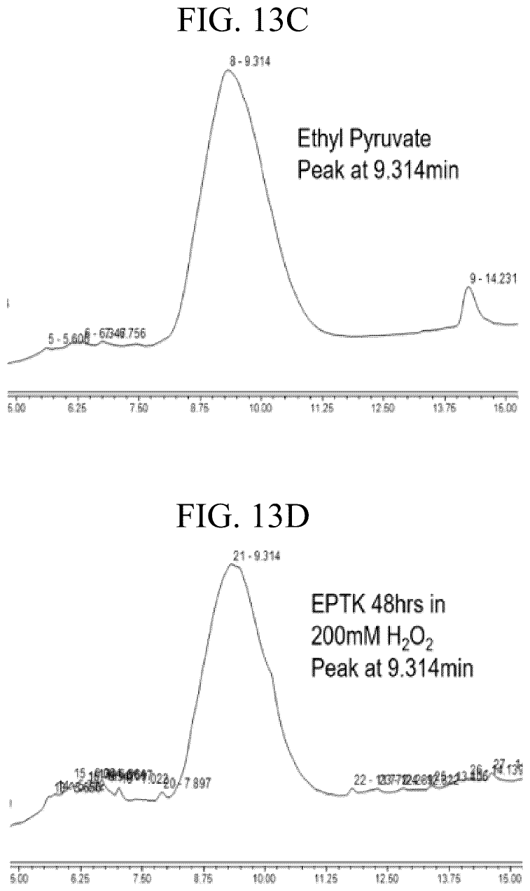

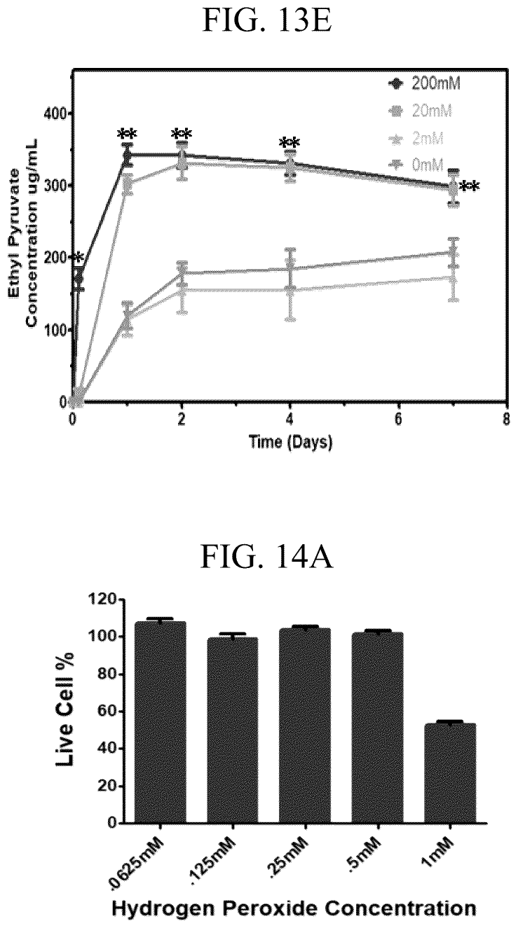

A new thioketal linker was synthesized containing the small molecule therapeutic ethyl pyruvate (FIG. 12). Successful creation of this new linker was confirmed by 1H nuclear magnetic resonance (NMR) as shown in FIG. 13A. When incubated in escalating doses of hydrogen peroxide (H₂O₂), a model reactive oxygen species (ROS), this linker also experiences dose-dependent degradation as expected due to its ROS-sensitive thioketal bond (FIG. 13B). To demonstrate this system's potential as a drug delivery vehicle, a sample of the ethyl pyruvate thioketal (EPTK) was incubated in 200 mM H₂O₂ for 48 hrs. As expected, intact ethyl pyruvate matching the original drug's character was released and quantified by high performance liquid chromatography as shown in FIGS. 13C and 13D. Moreover, incubating EPTK samples with increasing doses of ROS increased the rate of ethyl pyruvate release as well (FIG. 13E). Overall, these data demonstrate to potential for this system to both incorporate a ketone-containing drug molecule into a thioketal-based linker, and selectively release the intact drug upon treatment with ROS.

### Example 2

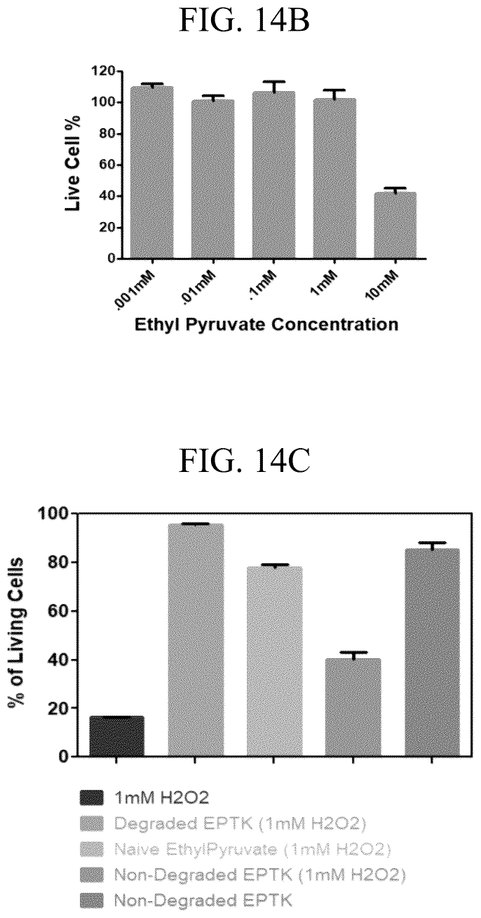

Released ethyl pyruvate was found to maintain its bioactivity as an antioxidant, cell-protective drug compound. As shown in FIGS. 14A and 14B, in vitro cytotoxicity profiles were first established for H₂O₂ and ethyl pyruvate with MC3T3-E1 pre-osteoblast cells. A dose of 1 mM H₂O₂ caused ˜50% cell death over 24 hrs in these cells, while ethyl pyruvate did not display any toxicity until a dose of 10 mM was reached. MC3T3-E1 cells were then exposed to various treatments to assess this system's bioactivity: 1 mM H2O2 as a toxic cell treatment, naïve ethyl pyruvate+1 mM H₂O₂ to establish antioxidant capacity of original drug, non-degraded EPTK to establish baseline toxicity of linker, degraded EPTK+1 mM H2O2 to see antioxidant effect of release ethyl pyruvate, and non-degraded EPTK+1 mM H2O2 to see if original linker offers any protective capacity. As shown in FIG. 14C, naïve ethyl pyruvate offers robust protection of cells from oxidative toxicity. Importantly, the non-degraded EPTK was found to be non-toxic and also offered some inherent antioxidant protection. However, ROS-degraded EPTK displayed substantial cellular protection from oxidative toxicity in this assay, demonstrating the promise of this system for the on-demand delivery of therapeutic compounds.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

What is claimed is:

1. A method of incorporating one or more linkers into a biomaterial with one or more ketone units comprising reacting a linker-containing material selected from the group consisting of thiol-containing compositions, thioketal-containing compositions, selenol-terminated compositions and combinations thereof with the biomaterial in the presence of an acid catalyst.

2. The method of claim 1 wherein the material is a thiol-containing composition.

3. The method of claim 2 wherein the thiol-containing composition is cysteamine.

4. The method of claim 1 wherein the material is a selenol-terminated composition.

5. The method of claim 4 wherein the selenol-terminated composition is 2-amino ethaneselenol.

6. The method of claim 1 wherein the material is a thioketal-containing composition.

7. The method of claim 6 wherein the thioketal-containing composition is cysteamine.

8. The method of claim 6 wherein the thioketal-containing composition is ethyl pyruvate.

9. The method of claim 1 wherein the linker in the linker-containing material is aromatic.

10. The method of claim 1 wherein the linker in the linker-containing material is cationic.

11. The method of claim 1 wherein the linker in the linker-containing material is anionic.

12. The method of claim 1 wherein the linker in the linker-containing material is a sulfonyl linker.

13. The method of claim 1 wherein the biomaterial is selected from the group consisting of polymeric coatings, tissue engineering scaffolds, hydrogels, and nanoparticles.

14. The method of claim 1 wherein the acid catalyst is selected from the group consisting of p-toluene sulfonic acid, tifluoroacetic acid, bismuth chloride, hydrochloric acid, and sulfuric acid.
